(19) United States
(12) Patent Application Publication
(10) Pub. No.: US 2018/0263519 A1
(43) Pub. Date: Sep. 20, 2018

(54) HEART RATE DETECTING MODULE AND METHOD

(71) Applicant: PIXART IMAGING INC., Hsin-Chu (TW)

(72) Inventor: REN-HAU GU, HSIN-CHU (TW)

(21) Appl. No.: 15/460,893

(22) Filed: Mar. 16, 2017

(51) Int. Cl. A61B 5/024; A61B 5/00

(52) U.S. Cl. CPC A61B 5/02427; A61B 5/7225; A61B 2576/00; A61B 2562/0238; A61B 2562/046; A61B 5/7278

(57) ABSTRACT

This instant disclosure provides a heart rate detecting module which includes an image sensor and a processor. The image sensor generates a plurality of image frames according to a light from a subject. The processor outputs a heart rate value based on a light intensity variance of the plurality of image frames. This instant disclosure further provides a heart rate detecting method which includes the following steps. A plurality of image frames are generated according to a light from a subject by an image sensor, and a heart rate value is outputted based on a light intensity variance of the plurality of image frames by a processor.

### BACKGROUND OF THE INVENTION

### 1. Field of the Invention

The instant disclosure relates to a detecting module and method; in particular, to a heart rate detecting module and method.

### 2. Description of Related Art

Generally, a PPG (photoplethysmogram) system is used to detect the heart rate according to the light brightness (light absorption) using a pulse oximeter which illuminates the skin and measures the changes in light absorption. When the heart contracts that has the maximum peripheral blood volume and light absorption and correspondingly has the minimum light brightness, and when the heart relaxes that has the maximum light brightness. So that a heartbeat can be determined. Accordingly, a heart rate detecting system has the dynamic range capable of detecting the maximum to minimum light intensity is necessary.

In the current PPG system, it usually includes a light source and a detector, and a pixel is used. However, single pixel usually has insufficient dynamic range and may resulted in too high noise ratio and so as to decrease the detection accuracy.

Therefore, how to reduce noise influence and upgrade the detection accuracy are important issues in the art.

### SUMMARY OF THE INVENTION

In order to overcome the abovementioned problem, this instant disclosure provides a heart rate detecting module which includes an image sensor and a processor. Via the image sensor including a CMOS (complementary metal oxide silicon) sensor array to generate a displacement information of light intensity gravity centers and the processor calculating a light intensity variance, a broad dynamic range can be obtained.

To achieve the abovementioned purpose, one of the embodiments of this instant disclosure provides a heart rate detecting module which includes an image sensor and a processor. The image sensor generates a plurality of image frames according to a light from a subject. The processor outputs a heart rate value based on a light intensity variance of the plurality of image frames.

Preferably, the light intensity variance is calculated by the processor via the displacement information of light intensity gravity centers of at least two of the plurality of image frames generated from the image sensor.

Preferably, the image sensor includes a sensor array which receives the light reflected from or passing through the subject to generate the plurality of image frames.

Preferably, the sensor array includes a plurality of pixels.

Another embodiment of this instant disclosure provides a heart rate detecting method which includes the following steps. A plurality of image frames are generated according to a light from a subject by an image sensor, and a heart rate value is outputted based on a light intensity variance of the plurality of image frames by a processor.

Yet another embodiment of this instant disclosure provides a heart rate detecting module which includes an image sensor and a processor. The image sensor generates a plurality of laser speckles according to a laser light from a subject. The processor outputs a heart rate value based on a change of at least one displacements of the plurality of laser speckles.

Yet another embodiment of this instant disclosure provides a heart rate detecting method which includes the following steps. An image sensor is used to generate a plurality of laser speckles according to a laser light from a subject. A processor is used to compare and analyze the plurality of laser speckles, to calculate at least one displacements of the plurality of laser speckles, and to output a heart rate value based on a change of the at least one displacements of the plurality of laser speckles.

This instant disclosure has the benefit that, via the heart rate detecting module includes the CMOS sensor array of the image sensor which can generate the displacement information of light intensity gravity centers and the displacements of laser speckles, and the processor can calculate the light intensity variance and changes of the displacements of the laser speckles, a broad dynamic range can be obtained. Therefore, the noise of detection signal can be reduced and the detection accuracy can be upgraded.

In order to further appreciate the characteristics and technical contents of the instant disclosure, references are hereunder made to the detailed descriptions and appended drawings in connection with the instant disclosure. However, the appended drawings are merely shown for exemplary purposes, rather than being used to restrict the scope of the instant disclosure.

### BRIEF DESCRIPTION OF THE DRAWINGS

FIG. 1 shows a schematic view of a heart rate detecting module of an embodiment about a light passing through a subject in the instant disclosure;

FIG. 2 shows a schematic view of a heart rate detecting module of an embodiment about a light being reflected from a subject in the instant disclosure;

FIG. 3 shows a flowchart of a heart rate detecting method of an embodiment in the instant disclosure;

FIG. 4 shows a schematic view of an image sensor of an embodiment in the instant disclosure;

FIG. 5 shows a schematic view of a placement of an image sensor and a light source;

FIG. 6A and 6B show heart rate detecting results measured by the heart rate detecting module of this instant disclosure; and

FIG. 7 shows a flowchart of a heart rate detecting method of the second embodiment in this instant disclosure.

### DETAILED DESCRIPTION OF THE PREFERRED EMBODIMENTS

Embodiments of heart rate detecting module and method disclosed in the instant disclosure are illustrated via specific examples as follows, and people familiar in the art may easily understand the advantages and efficacies of the instant disclosure by disclosure of the specification. The instant disclosure may be implemented or applied by other different specific examples, and each of the details in the specification may be applied based on different views and may be modified and changed under the existence of the spirit of the instant disclosure. The figures in the instant disclosure are only for brief description, but they are not depicted according to actual size and do not reflect the actual size of the relevant structure. The following embodiments further illustrate related technologies of the instant disclosure in detail, but the scope of the instant disclosure is not limited herein.

### First Embodiment

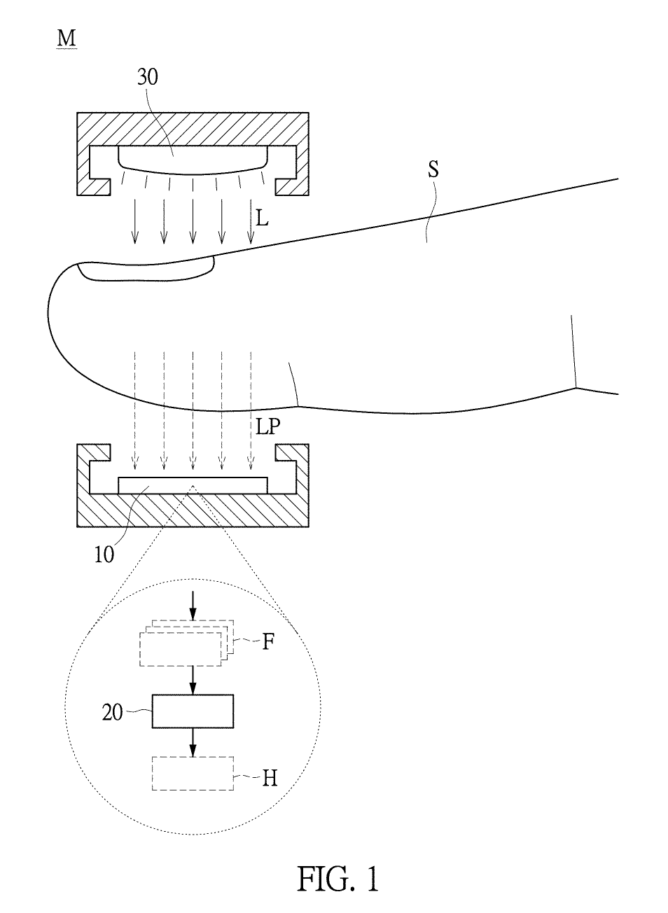

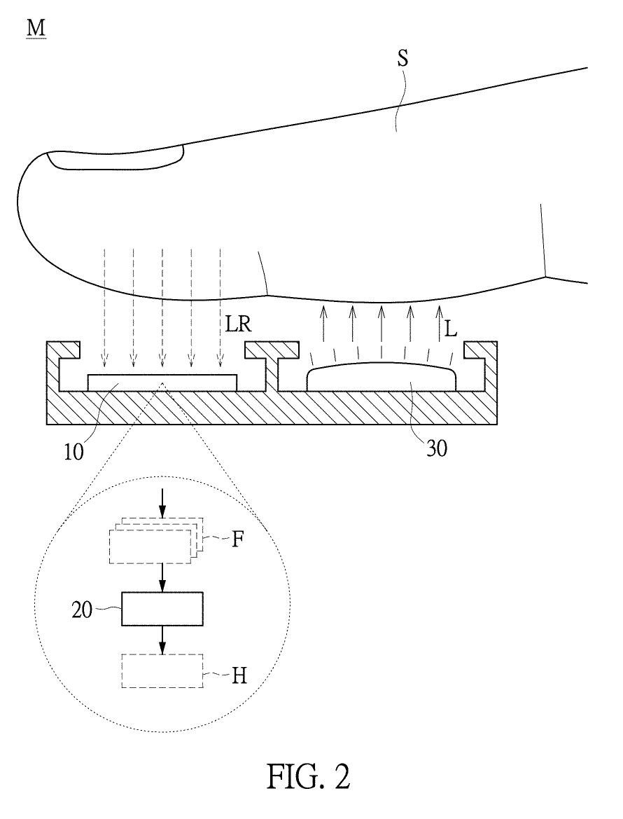

Please refer to FIG. 1, FIG. 2 and FIG. 4. FIG. 1 shows a schematic view of a heart rate detecting module of the first embodiment about a light passing through a subject in the instant disclosure, FIG. 2 shows a schematic view of a heart rate detecting module of the first embodiment about a light being reflected from a subject in the instant disclosure, and FIG. 4 shows a schematic view of an image sensor of the first embodiment in the instant disclosure. As shown in FIG. 1, the heart rate detecting module M of this embodiment includes an image sensor 10, a processor 20 and a light source 30. However, in other embodiments, the heart rate detecting module M may include a plurality image sensors 10, processors 20 and light sources 30, the number of the image sensor 10, the processor 20 and the light source 30 can be adjusted depending on requirements. In more details of FIG. 1, the image sensor 10 includes a sensor array which contains a plurality of pixels and is used to generate a corresponding plurality of image frames F. In this embodiment, the sensor array is a complementary metal oxide silicon (CMOS) sensor array 100. The CMOS sensor array 100 includes a plurality of pixels 1000 (as shown in FIG. 4), and the plurality of pixels 1000 of the CMOS sensor array 100 receive the light passing through the subject S (referred to passed light LP hereinafter) to generate an image frame F. In this embodiment, the processor 20 is a digital signal processor (DSP), and is used to output a heart rate value H. The light source 30 may be light-emitting diodes or laser lights, and is used to emit a light L toward to a subject S. The light L may have a limited bandwidth to improve the CMOS sensor array 100 sensing the light L. Furthermore, the processor 20 controls the light source 30, so that the light source 30 can keep lights on or intermittently lights on. For example, the processor 20 controls the light source 30 to emit the light L 20 times per second, in one embodiment, the CMOS sensor array 100 of the image sensor 10 samples in a rate (i.e. sample rate) sync to the flash rate of the light source 30 and therefore receives the passed light LP 20 times and generates 20 image frames F. In the embodiment of FIG. 1, the CMOS sensor array 100 of the image sensor 10 samples in the rate sync to the flash rate of the light source 30 to improve a sensing result, but it is not limited herein. In other embodiments, the CMOS sensor array 100 of the image sensor 10 may not sample in the rate sync to the flash rate of the light source 30.

As shown in FIG. 2, the image sensor 10 includes a sensor array which contains a plurality of pixels and is used to generate a plurality of image frames F as disclosed in the embodiment in FIG. 1. In this embodiment, the sensor array is a CMOS sensor array 100 which includes a plurality of pixels 1000 (as shown in FIG. 4), and the plurality of pixels 1000 of the CMOS sensor array 100 receive the light reflected from the subject S (referred to reflected light LR hereinafter) to generate an image frame F. The plurality of pixels 1000 of the CMOS sensor output intensity values to generate an image and generate a plurality of image frames F depends on a sample rate of the image sensor 10.

In this embodiment, the processor 20 is a DSP, and is used to output a heart rate value H. The light source 30 may be light-emitting diodes or laser lights, and is used to emit a light L toward to a subject S. Furthermore, the processor 20 controls the light source 30, so that the light source 30 can keep lights on or intermittently lights on. In the embodiment of FIG. 2, The CMOS sensor array 100 of the image sensor 10 samples in a rate (i.e. sample rate) sync to the flash rate of the light source 30 and therefore receives the reflected light LR and generates image frames F.

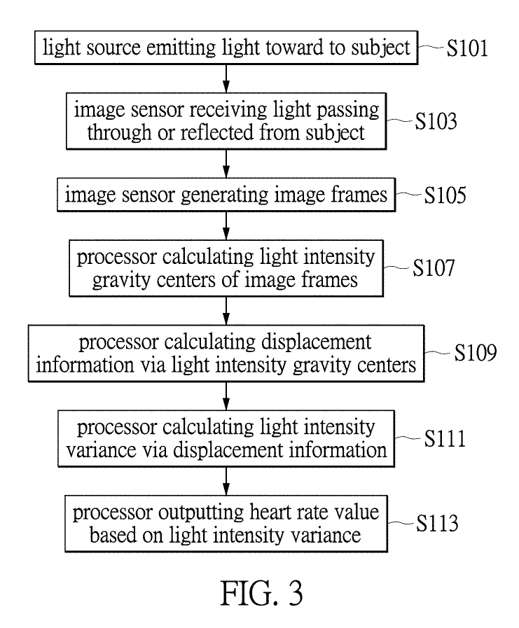

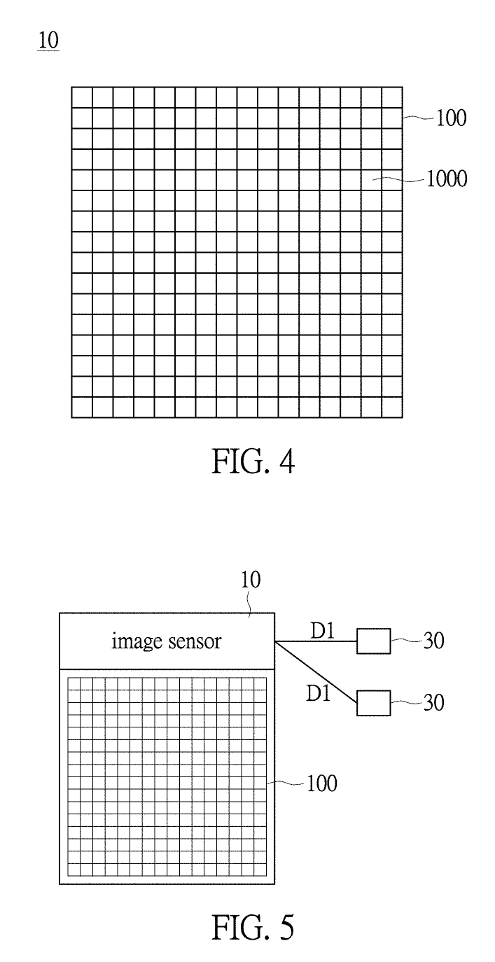

The instant disclosure focuses on calculating the heart rate according to a depth displacement (referred to displacement information hereinafter) of a surface (skin) being measured. The change of the depth from the surface to the CMOS sensor array 100 will cause a change of the light intensity gravity center of the passed light LP or the reflected light LR. Therefore, the displacement information can be calculated via a change of the light intensity gravity centers of the passed light LP or the reflected light LR, and a method for calculating the change of the light intensity gravity centers is described below. Specifically, please refer to steps S101 to S113 in FIG. 3 and FIG. 5. FIG. 3 shows a flowchart of a heart rate detecting method of the first embodiment in the instant disclosure, and FIG. 5 shows a schematic view of a placement of an image sensor and a light source. Firstly, as shown in FIG. 3 and FIG. 5, in the steps S101 and S103, the processor 20 controls the light source 30 to emit the light L toward to the subject S, the image sensor 10 is disposed at a distance D1 from the light source, in one embodiment the distance D1 is within the distance ranges from 1.8 mm to 4 mm, from 2.8 mm to 4 mm or from 3.8 mm to 4 mm In FIG. 5, the distance D1 is illustrated as 4 mm. In the next step S105, the image sensor 10 generates a plurality of image frames F according to the passed light LP or the reflected light LR. Then, in the steps S107 to S113, the processor 20 calculates positions of the light intensity gravity centers of at least two of the plurality of image frames F generated from the image sensor 10 (in step S107). According to the difference of two positions of the light intensity gravity centers, the displacement information of the light intensity gravity centers is calculated by the processor 20 (in step S109), and a light intensity variance is then calculated by the processor 20 (in step S111) via the displacement information. In addition, the displacement information contains an X displacement data, a Y displacement data and a photoplethysmography data. The displacement information is a difference between two positions of the light intensity gravity center in different times. Where the position of the light intensity gravity center can be determined by a coordinate of each pixel and corresponding intensity values, such as list in formula (I) as follows.

Σ(Pi×Ii)/ΣIi=PGC (I)

In the formula (I), Pi represents a corresponding coordinate of each of the plurality of pixels 1000, and contains the coordinate of X, and the coordinate of Y. The PGC can be determined by two-dimensional coordinate system (contains X and Y coordinate) but also can be determined by one-dimensional coordinate system (contains only X or Y coordinates), wherein the two one-dimensional PGC (X coordinate and Y coordinate) can be combined as the two-dimensional PGC. Ii represents an intensity of the passed light LP or the reflected light LR received by each of the plurality of pixels 1000. Σli represents a sum of intensities of the passed light LP or the reflected light LR received by the plurality of pixels 1000. PGC (position of gravity center) represents the light intensity gravity center of each captured image, wherein a displacement information is a difference value of two position of gravity centers of two frames. Finally, the processor 20 outputs the heart rate value H based on the displacement information of the light intensity gravity centers of the plurality of image frames F. In addition, there are various methods for calculating the light intensity gravity centers in prior arts, the aforementioned formula listed herewith is only one of them. However, the methods for calculating the light intensity gravity centers is not limited herein.

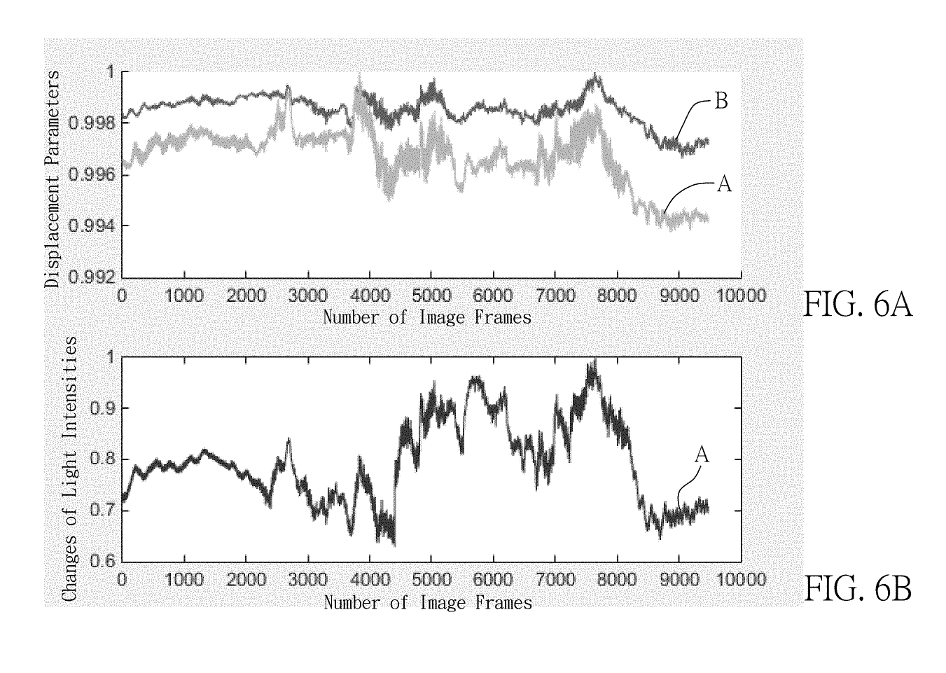

Please refer to FIGS. 6A and 6B. FIGS. 6A and 6B show heart rate detecting results of a motion activation measured by the heart rate detecting module M of this instant disclosure. In other words, the results of the heart rate detecting module M of this instant disclosure include FIG. 6A and FIG. 6B. In FIG. 6A, the horizontal axis represents number of image frames F, for example the number 1000 means the 1000-th frame captured by the image sensor 10, that are obtained from a runner's heart rates during running on a treadmill over time. In this figure, from number 0 to about number 2200, the runner was resting and started to run with a speed of 5 km/hr lasting for 1 minute (from number 2200 to about number 3600). As shown in from number 3600 to number 5500, the speed was increased to 9 km/hr lasting for 1 minute. Then, from number 5500 to number 6500, the speed was slowed down to 3 km/hr lasting for 1 minute. Next, the speed was increased to 7 km/hr lasting for 1 minute from number 6500 to number 7800, and after that the runner was resting (from number 7800). The longitudinal axis represents displacement parameters that are the pulse beating causing height changes of the detected skin, wherein the value from 0 to 1 represents the level of the height changes of the detected skin (value 0 represents no displacement and value 1 represents maximum displacement). Specifically, when the heart is beating, the blood would be outputted to generate vibrations, so as to make displacements of the skin and it is called displacement parameters. There are a displacement of X direction (the lower line: A line) and a displacement of Y direction (the upper line: B line) have been shown in FIG. 6A. In which, the maximum of the displacement parameter is 1, and there is the maximum displacement parameter at number 7800.

As shown in FIG. 6B, the conditions of heart rate detection is identical to aforementioned FIG. 6A, thus it is not repeated herein. The horizontal axis represents the number of image frames F, and the longitudinal axis represents changes of light intensity, wherein the value from 0 to 1 represents the level of the intensity changes of the captured image frame (value 0 represents no change and value 1 represents maximum change). Specifically, when the heart is beating, the blood would be outputted to generate vibrations, so as to make the changes of light intensity of the skin. There is only a PPG (photoplethysmogram) (A line) has been shown in FIG. 6B. In this figure, the maximum of the changes of light intensity is 1, and there is the maximum change of light intensity at number 7800.

According to above, it is showed that, the heart rate detecting module M of this instant disclosure can measure the X displacement, the Y displacement and the PPG, and the results can be compensated to decrease disturbing signals of motion (such as hands waving during running) and improve detection accuracy.

Since the heart rate detecting module M of this instant disclosure not only can generate the PPG data but also can generate the X displacement data and the Y displacement data, such that it can reduce interferences to output highly accurate heart rate results.

Accordingly, if a detecting module only can output a PPG data and it is like a traditional detecting module in prior arts, only one piece of pixel is used to receive the light, thus the dynamic range is insufficient, and the change of the PPG is limited, such that the noise of the heart rate detection signal is hard to be reduced.

Comparing to the prior arts, since the heart rate detecting module M of this instant disclosure has the CMOS sensor array 100 which is composed of a plurality of pixels 1000, each of the pixels 1000 receive the reflected light LR or the passed light LP and the results obtained therefrom can be summed up, thus a broad dynamic range can be obtained. Furthermore, the displacement information has two-dimensional information which contains an X displacement data, a Y displacement data, such that the noise of the heart rate detection signal (e.g., motion signal) can be effectively reduced to increase the accuracy of the heart rate result.

### Second Embodiment

A heart rate detecting module M of the second embodiment in this instant disclosure includes an image sensor 10 and a processor 20. The image sensor 10 generates a plurality of laser speckles according to a laser light from a subject S. The processor 20 outputs a heart rate value H based on a change of at least one displacements of the plurality of laser speckles.

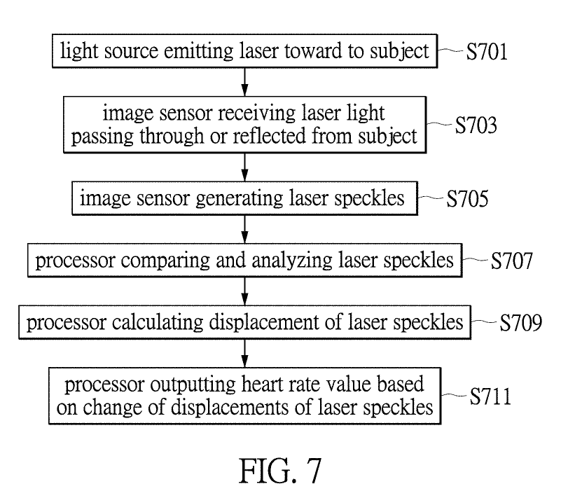

Please refer to FIG. 7. FIG. 7 shows a flowchart of a heart rate detecting method of the second embodiment in this instant disclosure. The heart rate detecting method of the second embodiment is a speckle pixel positioning method. Specifically, it includes the following steps, as shown in steps S701 to S711 in FIG. 7, a heart rate detecting method of the second embodiment in this instant disclosure includes the following steps. Firstly, in step S701 and step S703, a light source 30 is used to emit a laser light toward to a subject S, then an image sensor 10 receives the laser light passing through or reflected from the subject S (passed light LP and reflected light LR respectively). Next, in step S705, a plurality of laser speckles is generated according to the laser light from the subject S by an image sensor 10. Then, in step S707 and step S709, a processor 20 is used to compare and analyze the plurality of laser speckles, and to calculate changes of the plurality of laser speckles, that is at least one displacements of the plurality of laser speckles are calculated by the processor 20. Finally, the processor 20 outputs a heart rate value H based on a change or changes of the at least one displacements of the plurality of laser speckles.

In the second embodiment of this instant disclosure, except to the aforementioned heart rate detecting module M and the detecting method thereof, other technical features are the results obtained therefrom are identical to that of the first embodiment in this instant disclosure, thus it is not repeated herein.

In summary, this instant disclosure has the benefit that, via the heart rate detecting module includes the CMOS sensor array of the image sensor which can generate the displacement information of light intensity gravity centers and the displacements of laser speckles, and the processor can calculate the light intensity variance and changes of the displacements of the laser speckles, a broad dynamic range can be obtained. Therefore, the noise of detection signal can be reduced and the detection accuracy can be upgraded.

The descriptions illustrated supra set forth simply the preferred embodiments of the instant disclosure; however, the characteristics of the instant disclosure are by no means restricted thereto. All changes, alterations, or modifications conveniently considered by those skilled in the art are deemed to be encompassed within the scope of the instant disclosure delineated by the following claims.

What is claimed is:

1. A heart rate detecting module, comprising:
an image sensor which generates a plurality of image frames according to a light from a subject; and
a processor which outputs a heart rate value based on a light intensity variance of the plurality of image frames.

2. The heart rate detecting module as claimed in claim 1, wherein the light intensity variance is calculated by the processor via a displacement information of light intensity gravity centers of at least two of the plurality of image frames generated from the image sensor.

3. The heart rate detecting module as claimed in claim 1, wherein the image sensor generates the plurality of image frames according to the light which is reflected from or passing through the subject.

4. The heart rate detecting module as claimed in claim 1, further comprising a light source capable of emitting the light toward to the subject.

5. The heart rate detecting module as claimed in claim 1, wherein the image sensor includes a sensor array which receives the light reflected from or passing through the subject to generate the plurality of image frames.

6. The heart rate detecting module as claimed in claim 5, wherein the sensor array includes a plurality of pixels.

7. The heart rate detecting module as claimed in claim 4, wherein the processor controls the light source.

8. The heart rate detecting module as claimed in claim 6, wherein the plurality of pixels of the sensor array receives the light reflected from or passing through the subject.

9. The heart rate detecting module as claimed in claim 2, wherein the displacement information is a difference between two positions of the light intensity gravity center in different times, where the position of the light intensity gravity center is determined by a coordinate of each pixel and corresponding intensity values, listed in a formula as follows:
Σ(Pi×Ii)/ΣIi=PGC
wherein Pi represents a corresponding coordinate of each of the plurality of pixels, Ii represents an intensity of a reflected light or a passed light received by each of the plurality of pixels, ΣIi represents a sum of intensities of the reflected light or the passed light received by the plurality of pixels, and PGC represents the light intensity gravity center of each captured image.

10. The heart rate detecting module as claimed in claim 5, wherein the sensor array is disposed at a distance from the light source, and the distance ranges from 1.8 mm to 4 mm.

11. The heart rate detecting module as claimed in claim 4, wherein the source is light-emitting diodes or a laser light.

12. The heart rate detecting module as claimed in claim 1, wherein the processor is a digital signal processor.

13. The heart rate detecting module as claimed in claim 2, wherein the displacement information contains an X displacement data, a Y displacement data, and a photoplethysmography data.

14. The heart rate detecting module as claimed in claim 13, wherein the Pi contains one of the coordinate of X and the coordinate of Y

15. The heart rate detecting module as claimed in claim 13, wherein the Pi contains the coordinate of X and the coordinate of Y

16. A heart rate detecting method, comprising:
generating a plurality of image frames according to a light from a subject by an image sensor; and
outputting a heart rate value based on a light intensity variance of the plurality of image frames by a processor.

17. The heart rate detecting method as claimed in claim 16, wherein the light intensity variance is calculated by the processor via a displacement information of light intensity gravity centers of at least two of the plurality of image frames generated from the image sensor.

18. The heart rate detecting method as claimed in claim 16, wherein generating the plurality of image frames according to the light which is reflected from or passing through the subject.

19. The heart rate detecting method as claimed in claim 16, further comprising emitting the light toward to the subject by a light source.

20. The heart rate detecting method as claimed in claim 16, wherein the image sensor includes a sensor array which receives the light reflected from or passing through the subject to generate the plurality of image frames.

21. The heart rate detecting method as claimed in claim 20, wherein the sensor array includes a plurality of pixels.

22. The heart rate detecting method as claimed in claim 19, further comprising controlling the light source by the processor.

23. The heart rate detecting method as claimed in claim 21, wherein the plurality of pixels of the sensor array receives the light reflected from or passing through the subject.

24. The heart rate detecting method as claimed in claim 17, wherein the displacement information is a difference between two positions of the light intensity gravity center in different times, where the position of the light intensity gravity center is determined by a coordinate of each pixel and corresponding intensity values, listed in a formula as follows:
Σ(Pi×Ii)/ΣIi=PGC
wherein Pi represents a corresponding coordinate of each of the plurality of pixels, Ii represents an intensity of a reflected light or a passed light received by each of the plurality of pixels, ΣIi represents a sum of intensities of the reflected light or the passed light received by the plurality of pixels, and PGC represents the light intensity gravity center of each captured image.

25. The heart rate detecting method as claimed in claim 20, wherein the sensor array is disposed at a distance from the light source, and the distance ranges from 1.8 mm to 4 mm.

26. The heart rate detecting method as claimed in claim 19, wherein the source is light-emitting diodes or a laser light.

27. The heart rate detecting method as claimed in claim 16, wherein the processor is a digital signal processor.

28. The heart rate detecting method as claimed in claim 17, wherein the displacement information contains an X displacement data, and a Y displacement data.

29. The heart rate detecting method as claimed in claim 28, wherein the Pi contains one of the coordinate of X and the coordinate of Y.

30. The heart rate detecting method as claimed in claim 28, wherein the Pi contains the coordinate of X and the coordinate of Y.

31. A heart rate detecting module, comprising:
an image sensor which generates a plurality of laser speckles according to a laser light from a subject; and
a processor which outputs a heart rate value based on a change of at least one displacements of the plurality of laser speckles.

32. A heart rate detecting method, comprising:
generating a plurality of laser speckles according to a laser light from a subject by an image sensor;
comparing and analyzing the plurality of laser speckles by a processor;
calculating at least one displacements of the plurality of laser speckles by the processor; and
outputting a heart rate value based on a change of the at least one displacements of the plurality of laser speckles by the processor.
